(19) United States
(12) Patent Application Publication
(10) Pub. No.: US 2008/0132979 A1
(43) Pub. Date: Jun. 5, 2008

(54) Method of implanting a medical lead

(75) Inventor: Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/606,772

(22) Filed: Nov. 30, 2006

(51) Int. Cl. A61N 1/05

(52) U.S. Cl. 607/116

(57) ABSTRACT

A method for implanting one or more implantable medical leads of an electrical stimulation system proximate to an occipital region of a patient comprises utilizing a needle to define an insertion path through tissue of the patient to a target tissue site. The insertion path may be dilated to a size large enough to receive an implantable medical lead. An electrical test signal may be delivered to the patient via the needle, a guide wire or a dilator assembly during the implantation procedure in order to assess the efficacy of stimulation prior to implantation of the lead and/or to establish the location of the needle, guide wire and/or dilator assembly within the patient.

### TECHNICAL FIELD

The invention relates to medical device systems, and more particularly, to implanting an implantable medical lead of a medical device system within a patient.

### BACKGROUND

Electrical stimulation systems may be used to deliver electrical stimulation therapy to patients to treat a variety of symptoms or conditions such as chronic pain, tremor, Parkinson's disease, multiple sclerosis, spinal cord injury, cerebral palsy, amyotrophic lateral sclerosis, dystonia, torticollis, epilepsy, pelvic floor disorders, gastroparesis, muscle stimulation (e.g., functional electrical stimulation (FES) of muscles) or obesity. An electrical stimulation system typically includes one or more implantable medical leads coupled to an external or implantable electrical stimulator.

The implantable medical lead may be percutaneously or surgically implanted in a patient on a temporary or permanent basis such that at least one stimulation electrode is positioned proximate to a target stimulation site. The target stimulation site may be, for example, a nerve or other tissue site, such as a spinal cord, pelvic nerve, pudendal nerve, stomach, bladder, or within a brain or other organ of a patient, or within a muscle or muscle group of a patient. The one or more electrodes located proximate to the target stimulation site may deliver electrical stimulation therapy to the target stimulation site in the form of electrical signals.

Electrical stimulation of a peripheral nerve, such as stimulation of an occipital nerve, may be used to mask a patient's feeling of pain with a tingling sensation, referred to as paresthesia. Occipital nerves, such as a lesser occipital nerve, greater occipital nerve or third occipital nerve, exit the spinal cord at the cervical region, extend upward and toward the sides of the head, and pass through muscle and fascia to the scalp. Pain caused by an occipital nerve, e.g. occipital neuralgia, may be treated by delivering stimulation therapy to the occipital region via an implanted stimulation lead.

### SUMMARY

In general, the invention relates to a method for implanting one or more implantable medical leads of an electrical stimulation system proximate to an occipital region of a patient, which may be, for example, proximate to an occipital nerve or a trigeminal nerve. During the implantation procedure, a needle may be introduced into a patient to define an insertion path through tissue of the patient to the target tissue site. The insertion path may be dilated to a size large to receive the lead in order to easily introduce the lead into the patient. An electrical test signal may be delivered to the patient via the needle, a guide wire or a dilator assembly during the implantation procedure in order to assess the efficacy of stimulation (e.g., to determine whether the needle, guide wire or dilator assembly is properly positioned near the target tissue site) prior to implantation of the lead and/or to establish the depth and/or the location of the needle, guide wire and/or dilator assembly within the patient.

In one embodiment, the invention is directed toward a method comprising inserting a needle comprising a needle distal end into a patient, guiding the needle distal end to a target tissue site proximate to at least one of an occipital nerve or a trigeminal nerve of the patient, where the needle defines an insertion path through tissue of the patient, inserting a dilator into the patient through at least a portion of the insertion path, delivering an electrical test signal to the patient via at least one of the needle or the dilator, removing the needle from the patient, inserting an implantable medical lead into the patient through the dilator, and removing the dilator from the patient.

In another embodiment, the invention is directed toward a method comprising inserting a needle comprising a needle distal end into a patient, guiding the needle distal end to a target tissue site proximate to at least one of an occipital nerve or a trigeminal nerve of the patient, the needle defining an insertion path from skin of the patient to the target tissue site, inserting a guide wire through the needle to the target therapy delivery within the patient, removing the needle from the patient, inserting an implantable medical lead into the patient through the insertion path, removing the guide wire from the patient, and delivering an electrical test signal to the patient via at least one of the needle or the guide wire.

In yet another embodiment, the invention is directed toward a method comprising inserting a needle into a patient, guiding a needle distal end of the needle to a target tissue site proximate to at least one of an occipital nerve or a trigeminal nerve of the patient, the needle defining an insertion path through tissue of the patient, dilating the insertion path, delivering an electrical test signal to the patient via the needle, removing the needle from the patient, and inserting an implantable medical lead into the dilated insertion path.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram of a therapy system, which includes an electrical stimulator coupled to two stimulation leads that have been implanted in a body of a patient proximate to two target stimulation sites.

FIG. 2 is a schematic block diagram illustrating various components of an electrical stimulator and two implantable leads.

FIG. 3 illustrates the insertion of a needle into a patient to help facilitate implantation of a stimulation lead.

FIGS. 4A and 4B illustrate the needle of FIG. 3 in greater detail.

FIGS. 5A and 5B illustrate a dilator assembly.

FIG. 6A-6C illustrate the insertion of a dilator assembly over a needle to help facilitate implantation of a stimulation lead.

FIG. 7 is a flowchart illustrating an embodiment of the implantation method used to implant a lead without the aid of a needle.

FIG. 8 is a flowchart illustrating an embodiment of implanting a lead using a dilator sheath rather than a dilator assembly.

FIGS. 9A-9C illustrate the insertion of a guide wire into a needle and the insertion of a dilator assembly over the guide wire to help facilitate implantation of a stimulation lead.

FIG. 10 is a flowchart illustrating an embodiment of implanting a lead utilizing a guide wire.

FIG. 11 illustrates another embodiment, in which a lead is inserted into a patient around a guide wire.

FIG. 12 is a flow chart illustrating one embodiment of the implantation a lead utilizing the lead as a dilator.

FIG. 13 illustrates the insertion of a lead into a dilator sheath to help facilitate implantation of the lead.

FIG. 14 is a flowchart illustrating one embodiment of implanting a lead.

### DETAILED DESCRIPTION

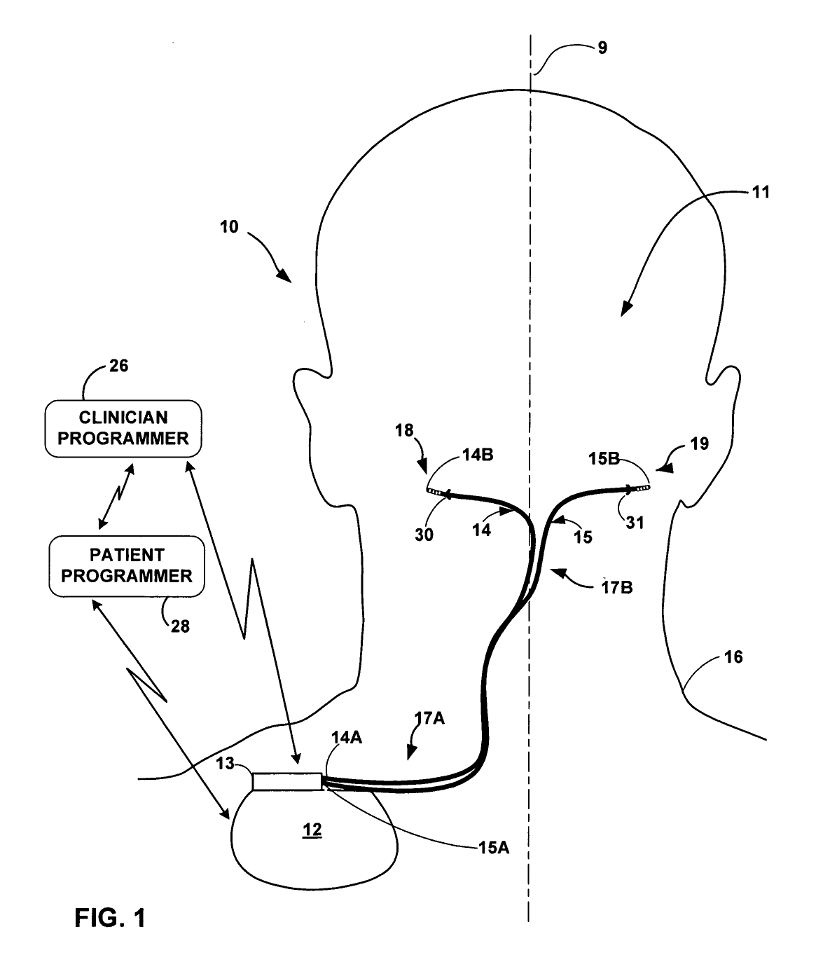

FIG. 1 is a schematic diagram of therapy system 10, which includes electrical stimulator 12 coupled to stimulation leads 14 and 15. In the example of FIG. 1, electrical stimulator 12 is implanted in patient 16 proximate to target stimulation sites 18 and 19. In one embodiment, target stimulation sites 18 and 19 are proximate to an occipital region 11 within patient 16. Occipital region 11 generally encompasses occipital nerve sites and trigeminal nerve sites of patient 16, which may be, for example, an occipital nerve (e.g., a greater occipital nerve, lesser occipital nerve, third occipital nerve), a trigeminal nerve, tissue adjacent to the trigeminal or occipital nerves, or a nerve branching from the occipital and/or trigeminal nerves. Thus, reference to an “occipital nerve” or a “trigeminal nerve” throughout the disclosure also includes branches of the occipital and trigeminal nerves, respectively. In addition, the therapy may be delivered to both an occipital nerve and trigeminal nerve by a single therapy system 10 or by separate therapy system (e.g., by separate electrical stimulators and leads).

Electrical stimulator 12 provides a programmable stimulation signal (e.g., in the form of electrical pulses or substantially continuous-time signals) that is delivered to target stimulation sites 18 and 19 by implantable medical leads 14 and 15, respectively, and more particularly, via stimulation electrodes carried by leads 14 and 15. Electrical stimulator 12 may also be referred to as a pulse or signal generator, and in the embodiment shown in FIG. 1, electrical stimulator 12 may also be referred to as a neurostimulator. In some embodiments, lead 14 and/or lead 15 may also carry one or more sense electrodes to permit neurostimulator 12 to sense electrical signals or other physiological parameters (e.g., blood pressure, temperature, etc.) from target stimulation site 18 and/or 19, respectively.

Proximal ends 14A and 15A of leads 14 and 15, respectively, may be both electrically and mechanically coupled to connection ports of connector block 13 of neurostimulator 12 either directly or indirectly (e.g., via a lead extension). In particular, conductors disposed in the lead body of each of leads 14 and 15 may electrically connect stimulation electrodes (and sense electrodes, if present) adjacent to distal ends 14B and 15B of leads 14 and 15, respectively, to neurostimulator 12.

In the embodiment of therapy system 10 shown in FIG. 1, target stimulation sites 18 and 19 are located within the patient's head (e.g., proximate to one or more occipital nerve) and on opposite sides of midline 9 of patient 16. Midline 9 is a schematic representation of the line that divides patient 16 into approximately equal and symmetrical left and right halves. Delivering therapy to two target tissue sites, such as sites 18 and 19, may be used to deliver therapy to two nerve branches that branch from the same nerve. Nerves may branch into left and right branches that extend to opposite sides of midline 9, and therapy is delivered to two nerve branches on opposite sides of midline 9 (such as at target tissue sites 18 and 19). Stimulation of two nerve branches on opposite sides of midline 9 may be referred to as bilateral stimulation. However, bilateral stimulation may also refer to stimulation of any two regions of patient 16 either sequentially or simultaneously. Delivering therapy after nerves branch, e.g., closer to the nerve endings, may allow more targeted therapy delivery with fewer side effects.

Stimulation of the occipital region 11 (i.e., in regions of patient 16 proximate to occipital nerves, a trigeminal nerve or other cranial sites) may help alleviate pain associated with, for example, chronic migraines, cervicogenic headaches, occipital neuralgia or trigeminal neuralgia.

Therapy system 10, however, is useful in other neurostimulation applications. Thus, in alternate embodiments, target stimulation sites 18 and 19 may be at locations proximate to any other suitable nerve in body of patient 16, which may be selected based on, for example, a therapy program selected for a particular patient. For example, in other embodiments, therapy system 10 may be used to deliver neurostimulation therapy to other areas of the nervous system, in which cases, lead 14 would be implanted proximate to the respective nerve(s). As one example, leads 14 and 15 may be implanted proximate to other nerves and/or structures of the head and neck of patient 16. For example, when therapy system 10 is used for stimulating a trigeminal nerve, target stimulation sites 18 and 19 may be on the side or front of the head of patient 16.

Accurate lead placement may affect the success of occipital nerve stimulation. If lead 14 and/or lead 15 is located too deep, i.e., anterior, in the subcutaneous tissue, patient 30 may experience muscle contractions, grabbing sensations, or burning. Such problems may additionally occur if lead 14 and/or lead 15 migrates after implantation. Furthermore, due to the location of implanted leads 14 and 15 on the back of the neck of patient 16, leads 14 and 15 may be subjected to pulling and stretching that may increase the chances of lead migration. For these reasons, leads 14 and 15 may include one or more fixation elements 30 and 31, respectively, to help prevent migration.

In the illustrated embodiment, leads 14 and 15 include tine-like fixation elements 30 and 31, respectively, which are configured to engage with surrounding tissue to substantially fix a position of leads 14 and 15. Fixation elements 30 and 31 may be expanded or activated by any suitable means. In some embodiments, fixation elements 30 and 31 may be restrained or otherwise prevented from premature fixation by a lead introducer, sheath, or other mechanism, prior to introduction into patient 16. Upon implantation into patient 16, fixation elements 30 and 31 may be expanded or activated by active or passive means. For example, in embodiments in which fixation elements 30 and 31 are tine-like structures, they may be expandable by elastic force such that fixation elements 30 and 31 automatically expand upon removal of the restraint mechanism.

Although fixation elements 30 and 31 are shown to be tine-like elements in the embodiment of FIG. 1, in other embodiments, fixation elements 30 and 31 may each be any suitable actively or passively deployed fixation element that helps prevent migration of leads 14 and 15 when leads 14 and 15 are implanted in patient 16, such as, but not limited to, one or more barbs, hooks, wire-like elements, adhesives (e.g., surgical adhesives), balloon-like fixation elements, tissue receiving cavities, pinning fixation elements, collapsible or expandable fixation structures, and so forth. In addition, fixation elements 30 and 31 may be formed in situ (i.e., after leads 14 and 15 are implanted in patient 16), such as by delivering a solidifying material (e.g., an adhesive or a hardenable structure material) to one or more exit ports along one or more surface of lead 14 and/or 15 to form fixation elements that extend from lead 14 and/or 15 to engage with surrounding tissue. Fixation elements 30 and 31 may be composed of any suitable biocompatible material, including, but not limited to, polymers, titanium, stainless steel, Nitinol, other shape memory materials, hydrogel or combinations thereof.

In some embodiments, fixation elements 30 and 31 are attached directly to leads 14 and 15. However, in other embodiments, fixation elements 30 and 31 may not be attached directly to leads 14 and 15, but may be carried by another apparatus that is attached to the leads 14 and 15, such as a sleeve or mounting band. An example of a mounting band is described in commonly-assigned U.S. Pat. No. 6,999,819, entitled “IMPLANTABLE MEDICAL ELECTRICAL STIMULATION LEAD FIXATION METHOD AND APPARATUS” and issued on Feb. 14, 2006.

In the illustrated embodiment, neurostimulator 12 is implanted in the back of patient 16 over the trapezius muscle (e.g., electrical stimulator 12 may be disposed within a surgically formed subcutaneous pocket formed near the trapezius muscle). Neurostimulator 12 may be inserted into patient 16 at incision site 17A. Leads 14 and 15 may also be inserted into patient 16 at incision site 17A and advanced (e.g., by tunneling) to target tissue sites 18 and 19, respectively. In this manner, neurostimulator 12, lead 14, and lead 15 may all be inserted using a single incision at incision site 17A. Alternatively, a second incision may be made at incision site 17B to facilitate implantation of leads 14 and 15 within patient 16 and positioning leads 14 and 15 with respect to target tissue sites 18 and 19 to achieve useful stimulation therapy or sensing. In alternative embodiments, neurostimulator 12 may be implanted at other suitable locations within patient 16, such as but not limited to, in a chest cavity, lower back, lower abdomen, or buttocks of patient 16.

Therapy system 10 also may include a clinician programmer 26 and a patient programmer 28. Clinician programmer 26 may be a handheld computing device that permits a clinician to program neurostimulation therapy for patient 16, e.g., using input keys and a display. For example, using clinician programmer 26, the clinician may specify stimulation parameters for use in delivery of electrical stimulation therapy. Clinician programmer 26 supports telemetry (e.g., radio frequency telemetry) with neurostimulator 12 to download neurostimulation parameters and, optionally, upload operational or physiological data stored by neurostimulator 12. In this manner, the clinician may periodically interrogate neurostimulator 12 to evaluate efficacy and, if necessary, modify the stimulation parameters.

Like clinician programmer 26, patient programmer 28 may be a handheld computing device. Patient programmer 28 may also include a display and input keys to allow patient 16 to interact with patient programmer 28 and neurostimulator 12. In this manner, patient programmer 28 provides patient 16 with an interface for control of neurostimulation therapy by neurostimulator 12. For example, patient 16 may use patient programmer 28 to start, stop or adjust neurostimulation therapy. In particular, patient programmer 28 may permit patient 16 to adjust stimulation parameters such as duration, amplitude, pulse width and pulse rate, within an adjustment range specified by the clinician via clinician programmer 28, or select from a library of stored stimulation therapy programs.

Neurostimulator 12, clinician programmer 26, and patient programmer 28 may communicate via cables or a wireless communication, as shown in FIG. 1. Clinician programmer 26 and patient programmer 28 may, for example, communicate via wireless communication with neurostimulator 12 using RF telemetry techniques known in the art. Clinician programmer 26 and patient programmer 28 also may communicate with each other using any of a variety of local wireless communication techniques, such as RF communication according to the 802.11 or Bluetooth specification sets, infrared communication, e.g., according to the IrDA standard, or other standard or proprietary telemetry protocols.

However, clinician programmer 26 and patient programmer 28 need not communicate wirelessly. For example, in other embodiments, programmers 26 and 28 may communicate via a wired connection, such as via a serial communication cable, or via exchange of removable media, such as magnetic or optical disks, or memory cards or sticks. Further, the clinician programmer 26 may communicate with patient programmer 28 via remote telemetry techniques known in the art, communicating via a local area network (LAN), wide area network (WAN), public switched telephone network (PSTN), or cellular telephone network, for example.

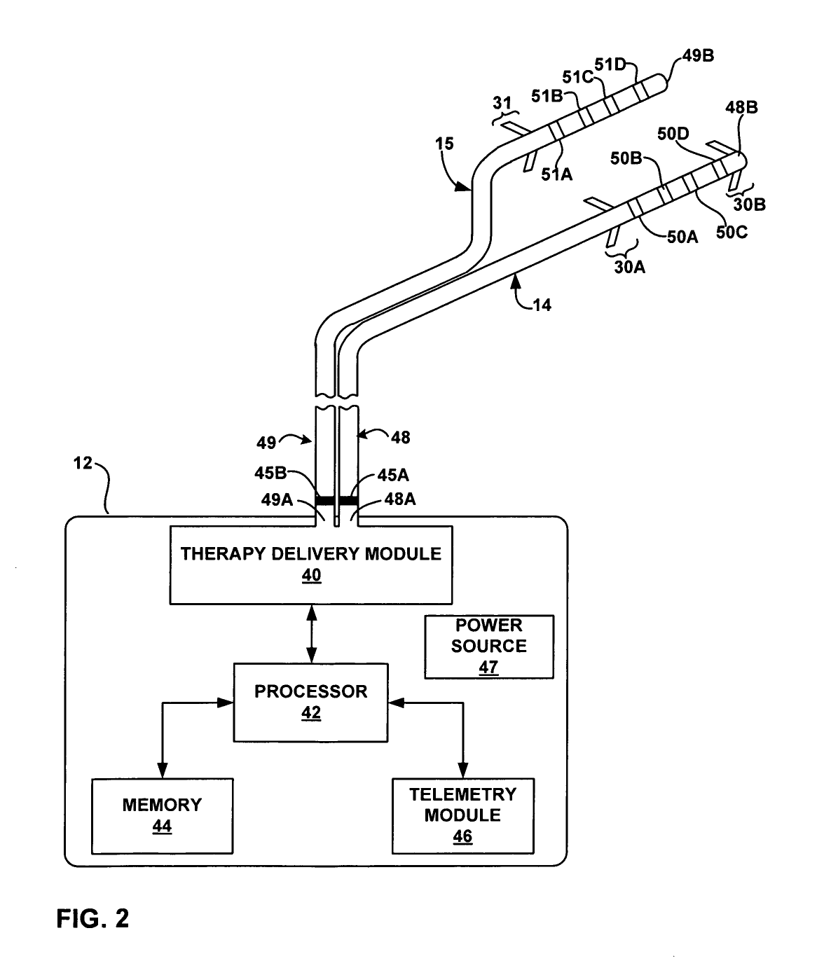

FIG. 2 is a block diagram illustrating various components of neurostimulator 12 and implantable leads 14 and 15 of therapy delivery system 10. Neurostimulator 12 includes therapy delivery module 40, processor 42, memory 44, telemetry module 46, and power source 47. In some embodiments, neurostimulator 12 may also include a sensing circuit (not shown in FIG. 2). Implantable lead 14 includes lead body 48 extending between proximal end 48A and distal end 48B. Similarly, implantable lead 15 includes lead body 49 extending between proximal end 49A and distal end 49B. Lead bodies 48 and 49 may be cylindrical or may be paddle-shaped (i.e., a “paddle” lead).

Electrodes 50A, 50B, 50C, and 50D (collectively “electrodes 50”) are disposed on lead body 48 adjacent to distal end 48B of lead body 48. Electrodes 51A, 51B, 51C, and 51D (collectively “electrodes 51”) are disposed on lead body 49 adjacent to distal end 49B of lead body 49. The configuration, type, and number of electrodes 50 and 51 illustrated in FIG. 2 are merely exemplary. In some embodiments, electrodes 50 and 51 may be ring electrodes. In other embodiments, electrodes 50 and 51 may be segmented or partial ring electrodes, each of which extends along an arc less than 360 degrees (e.g., 90-120 degrees) around the periphery of lead bodies 48 and 49, respectively.

In embodiments in which lead 14 is a paddle lead, electrodes 50 may extend along one side of lead body 48. Electrodes 50 extending around a portion of the circumference of lead body 48 or along one side of a paddle lead may be useful for providing an electrical stimulation field in a particular direction/targeting a particular therapy delivery site. For example, electrodes 50 may be disposed along lead body 48 such that the electrodes face toward nerves within the occipital region 11 of patient 16, or otherwise away from the scalp of patient 16. This may be an efficient use of stimulation because electrical stimulation of the scalp may not provide any or may provide minimal useful therapy to patient 16. In addition, the use of segmented or partial ring electrodes 50 may also reduce the overall power delivered to electrodes 50 by neurostimulator 12 because of the efficient delivery of stimulation to the targeted nerve(s) by eliminating or minimizing the delivery of stimulation to unwanted or unnecessary regions within patient 16. Electrodes 51 of lead 15 may also extend along one side of lead body 49 (if lead body 49 is paddle-shaped) or may extend around a portion of lead body 48, as described with respect to electrodes 50 of lead 14.

In embodiments in which electrodes 50 extend around a portion of the circumference of lead body 48 or along one side of a paddle lead, lead body 48 may include one or more orientation markers 45A proximate to proximal end 48A that indicate the relative location of electrodes 50. Orientation marker 45A may be a printed marker on lead body 48, an indentation in lead body 48, a radiographic marker, or another type of marker that is visible or otherwise detectable (e.g., detectable by a radiographic device) by a clinician. Orientation marker 45A may help a clinician properly orient lead 14 such that electrodes 50 face the desired direction (e.g., away from the scalp) when lead 14 is implanted within patient 16. For example, orientation marker 45A may also extend around the same portion of the circumference of lead body 48 or along the side of the paddle lead as electrodes 50. In this way, orientation marker 45A faces the same direction as electrodes 50, thus indicating the orientation of electrodes 50 to the clinician. When the clinician implants lead 14 in the patient, orientation marker 45 may remain visible to the clinician. Lead 15 may also include one or more orientation markers 45B.

As FIG. 2 illustrates, leads 14 and 15 include fixation elements 30A-B and 31. Although not shown in FIG. 1, lead 14 includes fixation elements 30A proximal to electrodes 50 and fixation elements 30B distal to electrodes 50. Fixation elements 30A and 30B may help locally fix electrodes proximate to target stimulation site 18 (FIG. 1). In other embodiments, lead 15 may also include fixation elements located both proximally and distally to electrodes 51, or alternatively, lead 14 may only include fixation elements distal to electrodes 51. In other embodiments, leads 14 and 15 may include fixation elements at any suitable location along the length of lead bodies 48 and 49 to fix lead bodies 48 and 49 at various points between proximal ends 48A, 49A and distal ends 48B and 49B. The “length” is generally measured from the respective proximal end 48A, 49A to the respective distal end 48B, 49B of lead bodies 48 and 49.

Neurostimulator 12 delivers stimulation therapy to target tissue sites 18 and 19 via electrodes 50 and 51, respectively, of leads 14 and 15. Electrodes 50 and 51 are electrically coupled to a therapy delivery module 40 of neurostimulator 12 via conductors within lead bodies 48 and 49, respectively. More specifically, proximal end 48A of lead body 48 includes contacts (not shown) to electrically couple electrodes 50 directly to connector 13 of neurostimulator 12 or indirectly to neurostimulator 12 (e.g., via a lead extension). Similarly, proximal end 49A of lead body 49 includes contacts (not shown) to electrically couple electrodes 51 directly to connector 13 of neurostimulator 12 or indirectly to neurostimulator 12 (e.g., via a lead extension). In one embodiment, an implantable signal generator or other stimulation circuitry within therapy delivery module 40 delivers electrical signals (e.g., pulses or substantially continuous-time signals, such as sinusoidal signals) to targets stimulation sites 18 and 19 (FIG. 1) via at least some of electrodes 50 and 51 under the control of a processor 42. The implantable signal generator may be coupled to power source 47. Power source 47 may take the form of a small, rechargeable or non-rechargeable battery, or an inductive power interface that transcutaneously receives inductively coupled energy. In the case of a rechargeable battery, power source 47 similarly may include an inductive power interface for transcutaneous transfer of recharge power.

The stimulation energy generated by therapy delivery module 40 may be formulated as neurostimulation energy, e.g., for treatment of any of a variety of neurological disorders, or disorders influenced by patient neurological response. The signals may be delivered from therapy delivery module 40 to electrodes 50 and 51 via a switch matrix and conductors carried by leads 14 and 15 and electrically coupled to respective electrodes 50 and 51.

Processor 42 may include a microprocessor, a controller, a DSP, an ASIC, an FPGA, discrete logic circuitry, or the like. Processor 42 controls the implantable signal generator within therapy delivery module 40 to deliver neurostimulation therapy according to selected stimulation parameters. Specifically, processor 42 controls therapy delivery module 40 to deliver electrical signals with selected amplitudes, pulse widths (if applicable), and rates specified by the programs. In addition, processor 42 may also control therapy delivery module 40 to deliver the neurostimulation signals via selected subsets of electrodes 50 or 51 with selected polarities. For example, electrodes 50 may be combined in various bipolar or multi-polar combinations to deliver stimulation energy to selected sites, such as nerve sites adjacent an occipital nerve, spinal column, pelvic floor nerve sites, or cranial nerve sites. Electrodes 51 may also be combined in various bipolar or multi-polar combinations to deliver stimulation energy to selected sites, such as nerve sites adjacent the spinal column, pelvic floor nerve sites, or cranial nerve sites.

Processor 42 may also control therapy delivery module 40 to deliver each signal according to a different program, thereby interleaving programs to simultaneously treat different symptoms or provide a combined therapeutic effect. For example, in addition to treatment of one symptom such as migraine headaches, neurostimulator 12 may be configured to deliver neurostimulation therapy to treat other symptoms such as back pain. In such an embodiment, electrodes 50 of lead 14 may be positioned to deliver stimulation therapy for treating one symptom, and electrodes 51 of lead 15 may be positioned to deliver stimulation therapy for treatment of another symptom.

Memory 44 of neurostimulator 12 may include any volatile or non-volatile media, such as a RAM, ROM, NVRAM, EEPROM, flash memory, and the like. In some embodiments, memory 44 of neurostimulator 12 may store multiple sets of stimulation parameters that are available to be selected by patient 16 via patient programmer 28 (FIG. 1) or a clinician via clinician programmer 26 (FIG. 1) for delivery of neurostimulation therapy. For example, memory 44 may store stimulation parameters transmitted by clinician programmer 26 (FIG. 1). Memory 44 also stores program instructions that, when executed by processor 42, cause neurostimulator 12 to deliver neurostimulation therapy. Accordingly, computer-readable media storing instructions may be provided to cause processor 42 to provide functionality as described herein.

In particular, processor 42 controls telemetry module 46 to exchange information with an external programmer, such as clinician programmer 26 and/or patient programmer 28 (FIG. 1), by wireless telemetry. In addition, in some embodiments, telemetry module 46 supports wireless communication with one or more wireless sensors that sense physiological signals and transmit the signals to neurostimulator 12.

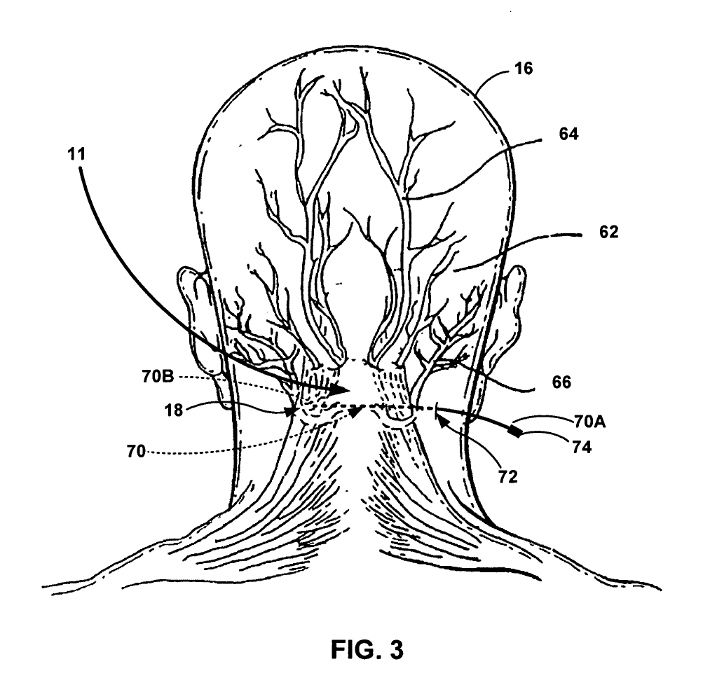

FIGS. 3-8 illustrate various stages in the implantation of lead 14 of therapy delivery system 10. A similar process may be used to implant lead 15. As shown in FIGS. 3-8, lead 14 may be implanted proximate to an occipital region 11 of patient 16 for stimulation of one or more occipital nerves. In particular, lead 14 may be implanted proximate to lesser occipital nerve 62, greater occipital nerve 64, and/or third occipital nerve 66. Alternatively, leads 14 and 15 may be implanted proximate to a trigeminal nerve located on the side or front of the patient's head (not shown in FIGS. 3-8).

Prior to beginning implantation of lead 14, a local anesthetic may be applied to anesthetize the area where stimulation lead 14 will be implanted, such as posterior to occipital region 11. Since embodiments of the implantation method permit the use of a local anesthetic, patient 16 may be treated on an out-patient basis, which may reduce costs over in-patient care and reduce recovery time. Also, by using local anesthesia, as opposed to general anesthesia, the implanting clinician may use patient's 16 conscious sensory response to stimuli (such as trial stimulation pulses) to aid in placing stimulation lead 14. Using patient's 16 conscious sensory response during placement of stimulation lead 14 may allow accurate placement of lead 14, may reduce the potential for an ineffective therapy, and more reduce the potential for patient 16 injury caused by a misplaced lead. In other embodiments, other forms of anesthesia can be used, such as general anesthesia.

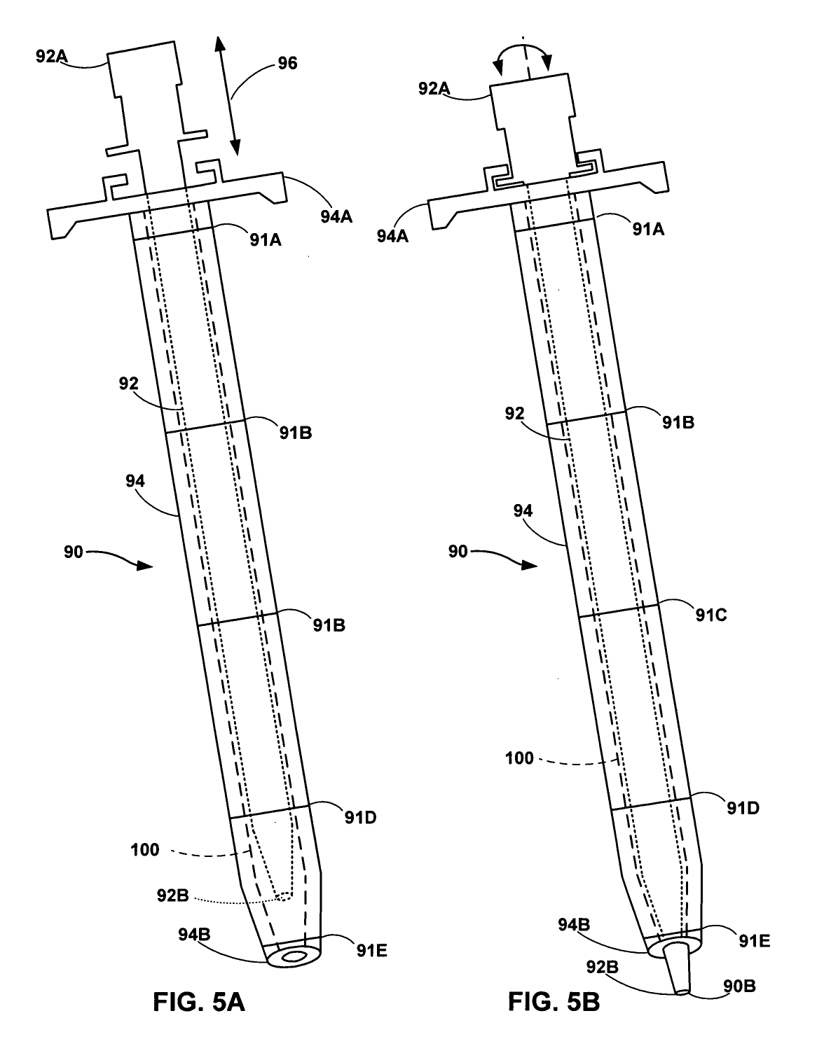

Once patient 16 has been anesthetized, needle 70 may be percutaneously introduced into patient 16 at entry point 72, as illustrated in FIG. 3, which may be created with a distal end 70B of needle 70, and guided to target tissue site 18. Alternatively, a small incision may be made to define entry point 72 for needle 70. The process of inserting and guiding needle 70 may involve the subcutaneous placement of lead 14 transversely across one or more occipital nerves 62, 64, and/or 66 that are causing patient 16 to experience pain. In the illustrated embodiment, needle 70 is introduced into the subcutaneous tissue, superficial to the fascia and muscle layer but below the skin. Occipital nerves 62, 64, and 66 are located within the cervical musculature and overlying fascia, and as a result, needle 70 and, eventually, dilator assembly 90 (shown in FIGS. 5A and 5B) and lead 14 are inserted superior to occipital nerves 62, 64, and 66. That is, in one embodiment, needle 70 is introduced into the fascia layer of patient 16 such that needle 70 is between the skin of patient 16 and target stimulation site 18 (FIG. 1). The approximate location of target tissue site 18 may be found using anatomical landmarks, fluoroscopy, or x-ray imaging. In order to locate the specific nerve causing pain, a clinician may palpate the area of pain.

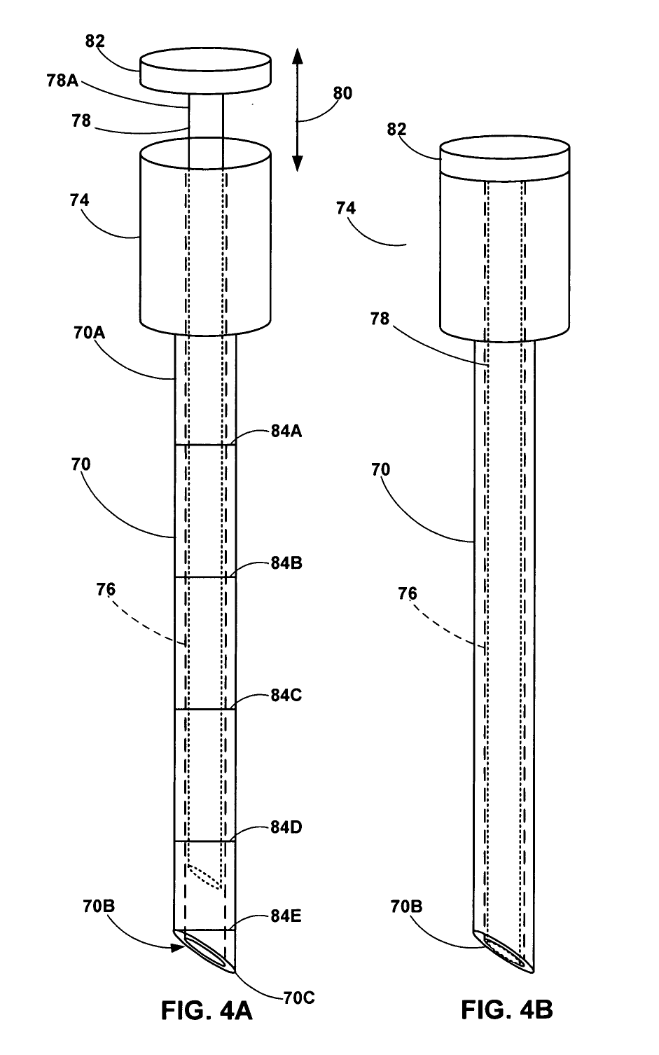

Needle 70 includes hub 74 located on proximate end 70A. Hub 74 may aid in handling and inserting needle 70. Additionally, hub 74 may be configured to connect to a syringe for injection of local anesthesia. In some embodiments, hub 74 may be removed from needle 70, for example, by using a cutting tool or any other appropriate means. Additionally, needle 70 may define an inner opening, referred to as lumen 76 (shown in FIGS. 4A and 4B), extending through hub 74 to distal end 70B of needle 70. In other embodiments, needle 70 may take a variety of other forms. For example, needle 70 may or may not include a hub 74 and/or a lumen 76.

Needle 70 has a relatively small outer perimeter (e.g., a diameter) in order to provide a minimally invasive apparatus for defining an insertion path from entry point 72 at the skin of patient 16 to target stimulation site 18. In some embodiments, an outer perimeter of needle 70 is smaller than the outer diameter of lead 14. When a clinician is implanting lead 14 within patient 16, the clinician may require more than one try to find an optimal target stimulation site 18. For example, the clinician may withdraw and reinsert needle 70 one or more times. Thus, it may be desirable to utilize the relatively small needle 70 to locate target stimulation site 18 and define the insertion path through tissue of patient 16 in order to minimize the invasiveness of the implantation procedure.

The size of needle 70 is selected based upon the needs of the patient 16 and the application of lead 14. In some embodiments, such as when lead 14 is implanted proximate to occipital region 11 of patient 16, needle 70 may have an outer diameter in the range of about 26 gauge (0.46 mm) to about 12 gauge (2.80 mm).

FIG. 4 illustrates needle 70 in further detail. Lumen 76 of needle 70 may be sized and configured to accommodate stylet 78. Stylet 78 may be inserted into needle 70 in the longitudinal direction (depicted by arrows 80) via hub 74. Additionally, stylet 78 may have a stylet hub 82 on proximal end 78A to aid in the handling of stylet 78. Stylet 78 may, but need not be, positioned inside lumen 76 of needle 70 when needle 70 is inserted into patient 16. When stylet 78 is disposed within lumen 76 of needle 70, tissue may be at least partially prevented from entering lumen 76 as needle 70 is advanced through tissue to target stimulation site 18. This may reduce tissue coring and undue damage to tissue or nerves by pushing tissue to the sides of needle 70 rather than into lumen 76.

Needle 70 may include radio-opaque markers 84A-84E that are detectable by imaging techniques, such as fluoroscopic imaging or x-ray imaging. In other embodiments, markers 84A-84E may be visible without the aid of imaging techniques. For example, markers 84A-84E may be printed markings (e.g., lines, text or graphical symbols) on needle 70, an indentation in needle 70 or another type of marker that is visible or otherwise detectable (e.g., detectable by a radiographic device) by the clinician. Markers 84A-84E may be helpful for maneuvering needle 70 relative to target tissue site 18 when inserting needle 70 within patient 16. For example, marker 84E near distal end 70B of needle 70 may indicate the depth of needle 70 and the relative location of distal end 70B of needle 70 as the clinician guides proximal distal end 70B to target stimulation site 18 in order to implant electrodes 50 of lead 14 proximate to target stimulation site 18.

Additionally each one of markers 84A-84E may represent a predetermined location on needle 70 such that markers 84A-84E may aid in determining needle 70 depth. As a clinician advances needle 70 into patient 16, the clinician may use markers 84A-84E to indicate how deep needle 70 has been introduced into patient 16. For example, in one embodiment, each marker 84A-84E may be placed at one centimeter (cm) intervals from distal end 70B of needle 70 (e.g., marker 84E=1 cm, marker 84D=2 cm, and so forth). If markers 84A and 84B are visible to the clinician, the clinician may determine about four centimeters of needle has been advanced into patient 16. Radio-opaque markers 84A-84E, as well as other types of markers, such as other types of radiographic and/or visible markers, may also be employed to assist a clinician during the introduction and withdrawal of needle 70 from patient 16.

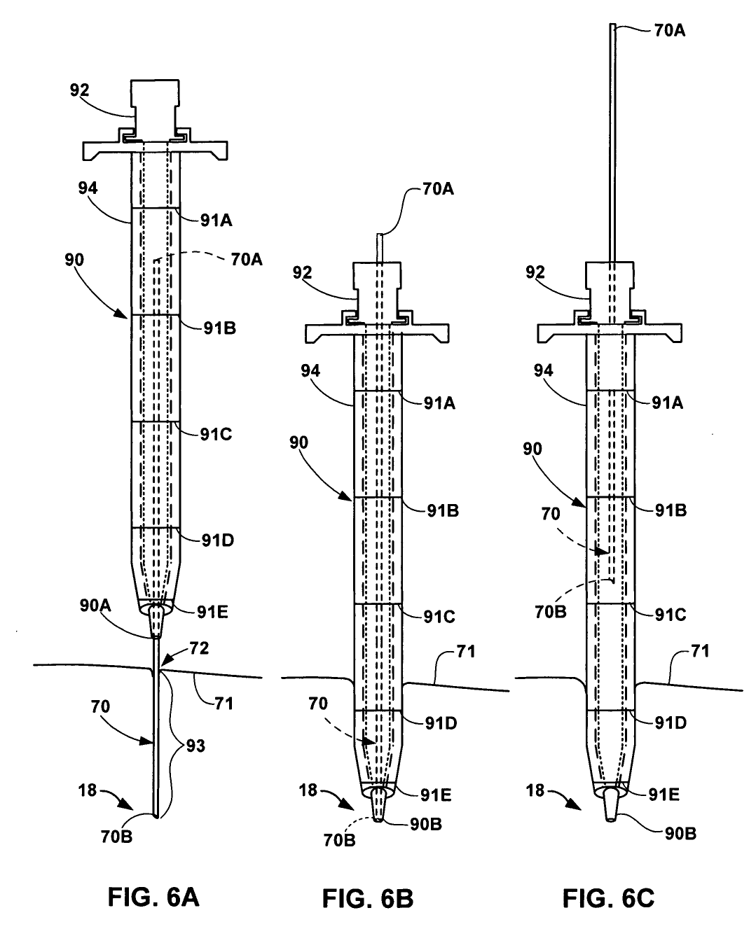

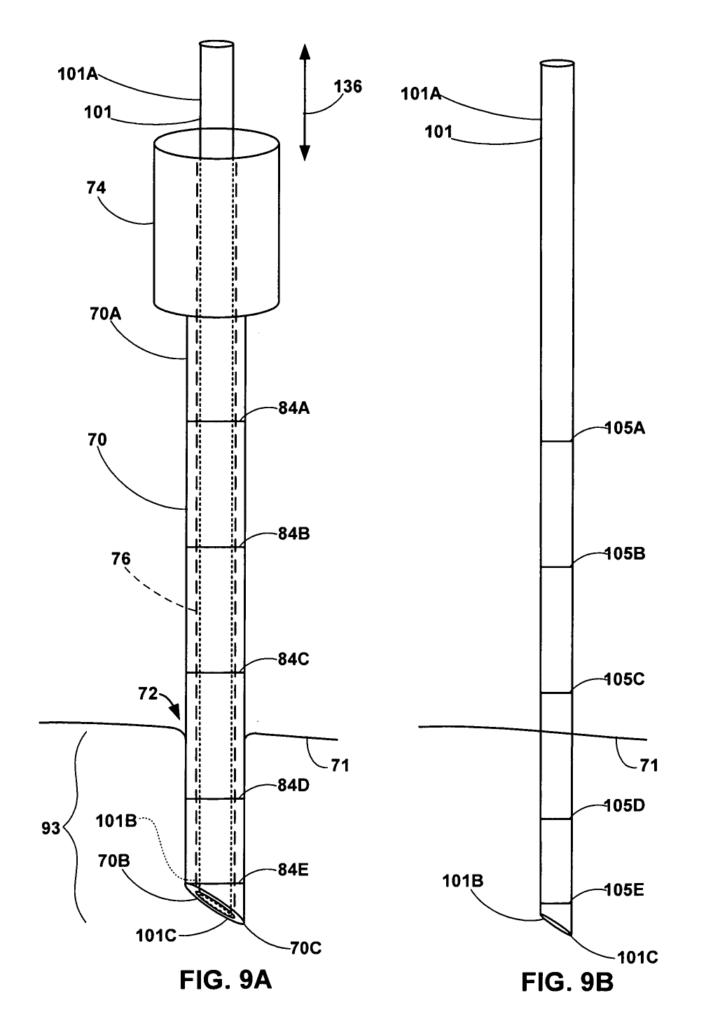

When guiding needle 70, the location/position of needle 70 can be determined by a variety of means. In one embodiment, an electrical signal is applied to needle 70 to evoke a response from patient 16, such as, for example, a motor or sensory response. For example, a portion of distal end 70B of needle 70, such as distal tip 70C, may be conductive. Additionally, a portion of proximal end 70A, such as hub 74, may be conductive and electrically connected (e.g., via an electrical conductor or an electrically conductive portion of needle 70) to the electrically conductive portions of distal end 70B to allow a trial electrical to be electrically connected to the conductive portion of proximal end 70A (e.g., hub 74). The remainder of needle 70 may be electrically insulated. In this manner, a trial stimulator stimulation signal (i.e., an “electrical test signal”) may be provided to hub 74 (or another electrically conductive portion of distal end 70B of needle 70) and travel to distal tip 70C to evoke a response from patient 16 to determine if needle 70 is properly positioned proximate to target stimulation site 18. The electrical test signal may also be used to determine whether patient 16 will likely benefit from stimulation. The patient response may be indicative whether the stimulation is felt, the efficacy of the stimulation, whether the stimulation results in any side effects, and so forth. Once needle 70 is in position at target tissue site 18, needle 70 can remain in the position to serve as a guide for dilator assembly 90 (FIGS. 5A-6C) or, alternatively, a guide wire 101 (shown and described with reference to FIGS. 9A and 9B).

In embodiments in which lead 14 is placed across midline 9 (FIG. 1) to achieve bilateral stimulation of occipital region 11, the clinician may gradually pull back needle 70 to provide electrical test signals to more than one target stimulation site within patient 16. For example, the clinician may observe markers 84A-E to retract needle 70 from patient with regularity and control. Upon confirming that needle 70 is properly positioned across the desired regions of stimulation across midline 9, the clinician may further insert needle 70 to a most distal target stimulation site within patient 16 in order to correctly position electrodes 50 across the desired region of stimulation.

FIGS. 5A-5B illustrate dilator assembly 90, which includes markers 91A-91E, which may be, for example, radio-opaque, fluoroscopic markers, or markers visible without the aid of a detection device. Dilator assembly 90 further includes dilator body 92 and dilator sheath 94. Dilator body 92 may be inserted into and removed from dilator sheath 94 along the longitudinal axis of dilator sheath 94 (represented by arrows 96). Dilator assembly 90 may be used to dilate the insertion path defined by needle 70 (or in some embodiments, the insertion path determined by a guide wire, which is described below). As previously discussed, needle 70 typically has a smaller outer perimeter than lead 14. As a result, it may be difficult in some embodiments to insert lead 14 into patient through the insertion path defined by needle 70. Dilator sheath 94 has an inner perimeter that is configured to receive lead 14. Accordingly, dilator assembly 90 may be used to dilate the insertion path defined by needle 70 to a sufficient size to introduce lead 14 into patient 16.

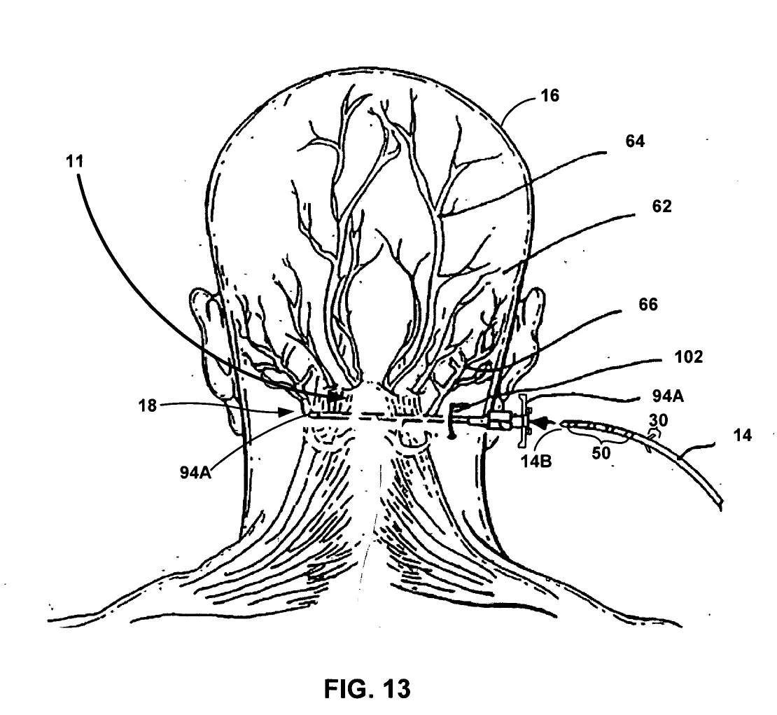

Depth markers 91A-91E may be disposed on dilator sheath 94 in order to help facilitate radiographic imaging when dilator sheath 94 is introduced into the body of patient 16. Depth markers 91A-91E may correlate with markers 84A-E on needle 70 in order to help correlate the relative positions of needle 70 and dilator assembly 90 when dilator assembly 90 is disposed at least partially around needle 80. For example, a clinician may determine that distal end 90B of dilator assembly 90 is aligned with distal end 70B of needle 70 when marker 84A of needle 70 is aligned with marker 91A on dilator sheath 94. In some embodiments, depth markers 91A-91E may be about one centimeter or about one-half centimeter bands or numerals or other indicia that indicate the depth of the exposed marker to a clinician. One or more depth markers 91A-91E on the surface of dilator sheath 94, such as depth marker 91A, may be visible at the skin incision site (e.g., at the site of incision 102 of FIG. 13) such that the depth of dilator assembly 90 may be read or otherwise determined without radiographic or other imaging techniques. Most distal marker 91E may be spaced from distal tip 94B of dilator sheath 94 to indicate a predetermined depth of distal tip 92B of dilator body 92 protruding distally from distal tip 94B of dilator sheath 94 when dilator assembly 90 is assembled, as shown in FIG. 5B.

Dilator body 92 may be conductive, and dilator sheath 94 may be non-conductive and electrically insulating. When assembled, as shown in FIG. 5B, distal end 92B of dilator body 92 extends out of distal end 94B of dilator sheath 94 and is electrically exposed. Electrical stimulation of occipital region 11 (FIG. 1) to test placement of distal end 92B of dilator body 92 may take place through dilator body 92 while dilator sheath 94 is in place.

Dilator sheath 94 comprises a central opening, referred to as dilator sheath lumen 100. Dilator sheath lumen 100 has a diameter sufficient to accommodate dilator body 92. Additionally, the diameter of dilator sheath 94 is sized to accommodate lead 14 when dilator body 92 is removed from dilator sheath 94. Both dilator sheath 94 and dilator sheath lumen 100 extend from proximal end 94A to distal end 94B of dilator sheath 94.

As FIGS. 6A-6C illustrate, dilator assembly 90 may be inserted over needle 70 and advanced to target tissue site 18 over at least a portion of the insertion path 93 defined by needle 70 from entry point to target tissue site 18. FIG. 6A illustrates needle 70, which has been introduced through skin 71, such that distal end 70B of needle 70 is positioned proximate to target tissue site 18. Proximal end 70A (shown in phantom) of needle extends away from the entry point 72 and away from the skin 71 of patient 16. Dilator body 92 is introduced around needle 70, and needle 70 is introduced into a lumen within dilator body 92. Prior to insertion of dilator assembly 90, stylet 78 may be removed from needle 70 in the longitudinal direction (represented by arrows 80 in FIG. 4A). Additionally, hub 74 may be removed from needle 70 in order to allow dilator assembly 90 to be inserted over needle 70. Hub 74 may be removed from needle 70 using a cutting tool or any other appropriate means.

An incision (e.g., incision 102 of FIG. 13) may be made proximate to entry site 72 to allow entry of dilator assembly 90 into patient 16. The incision may, for example, enlarge entry site 72 to permit entry of dilator assembly 90, which has a larger diameter than needle 70 in order to receive dilator assembly 90. In one embodiment, the incision may be about 5 cm wide. As illustrated in FIG. 6B, dilator assembly 90 may be advanced to target tissue site 18 using needle 70 as a guide. Once distal end 90B of dilator assembly 90 is aligned with distal end 70B of needle 70, needle 70 may be withdrawn from patient 16 while keeping distal end 90B of dilator assembly 90 positioned at target tissue site 18, as shown in FIG. 5C. A clinician may confirm that distal end 90B of dilator assembly 90 is aligned with distal end 70B of needle 70 by, for example, aligning markers 91A or 91B with one of markers 84A-E on needle 70. Alternatively, fluoroscopy may be used to determine the location of distal end 90B of dilator assembly 90 with respect to distal end 70B of needle 70. Yet another alternative, an electrical test signal may be delivered to distal end 90B of dilator assembly 90 to evoke a response from patient 16. In embodiments in which an electrical test signal was delivered to distal end 70B of needle 70, the patient responses could be compared to verify that the same response is maintained. When removing needle 70 from dilator assembly 90, care should be taken to avoid displacing dilator assembly 90.

In an alternative embodiment, instead of utilizing needle 70, an incision (e.g., incision 102 of FIG. 13) may be made near target stimulation site 18 and dilator assembly 90 may be directly advanced to target stimulation site 18 without the aid of needle 70.

After distal end 90B of dilator assembly 90 has been properly placed with respect to target tissue site 18, dilator body 92 may be removed from dilator sheath 94. As dilator body 92 is removed, the depth marker on dilator sheath 94 surface exposed at the skin incision site (e.g., at the site of incision 102 of FIG. 13), such as depth marker 94A, may be observed to ensure that dilator sheath 94 is not inadvertently advanced or withdrawn as dilator body 92 is withdrawn. Dilator sheath lumen 100 may be sized to sufficiently receive lead 14. In this manner, dilator sheath lumen 100 of dilator sheath 94 defines a passageway through which lead 14 may be advanced to target tissue site 18.

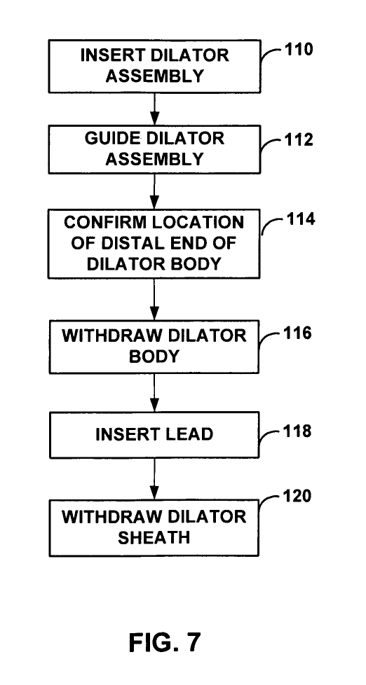

FIG. 7 is a flowchart illustrating one embodiment of the method for implanting lead 14 without the aid of needle 70. Dilator assembly 90 is inserted into patient 16 (110) and guided to target stimulation site 18 (112) to define an insertion path through tissue of patient 16. The location of distal end 90B of dilator assembly 90 is confirmed using one or more imaging techniques, electrical test signals delivered via dilator body 92 or another suitable means (114). Once the position of dilator assembly 90 is confirmed, dilator body 92 is removed from dilator sheath 94 and from patient 16 (116). Next, lead 14 is inserted in patient 16 through dilator sheath 94 (118), which defines an inner passageway that traverses the insertion path defined by dilator assembly 90. Dilator sheath 14 is sized to receive lead 14. Once lead 14, and particularly, electrodes 50 of lead 14, has been properly placed proximate to target stimulation site 18, dilator sheath 94 is removed from patient 16 (120). The location of electrodes 50 with respect to target tissue site 18 may be determined, for example, by providing an electrical test signal to electrodes 50 and receiving patient feedback that indicates the location of electrodes 50 (e.g., indicates whether electrodes 50 are placed to achieve sufficient paresthesia coverage.

In an alternate embodiment, one or more dilator sheaths 94 may be used to create an opening appropriately sized for insertion of lead 14 instead of using dilator assembly 90. Dilator sheath 94 may be metal or plastic and may have an outer diameter in the range of about 0.33 millimeters (mm) (5 French) to about 4.00 mm (14 French), selected based upon the size of stimulation lead 14 to be implanted. In some embodiments, multiple dilator sheaths of varying diameters are used in sequence from a smaller diameter to a larger diameter to achieve the desired dilation while controlling tissue trauma. For example, a first dilator sheath may be inserted over needle 70 or guide wire 101, and needle 70 or guide wire 101 may be removed once the first dilator sheath is properly placed. Then a second dilator sheath with a larger diameter than the first may be inserted over the first dilator sheath, and the first dilator sheath may be removed once the second dilator sheath is properly placed. This process may be repeated until a dilator sheath appropriately sized to pass stimulation lead 14, such as 2.6 mm (8 French) sized dilator sheath, has been properly inserted into patient 14.

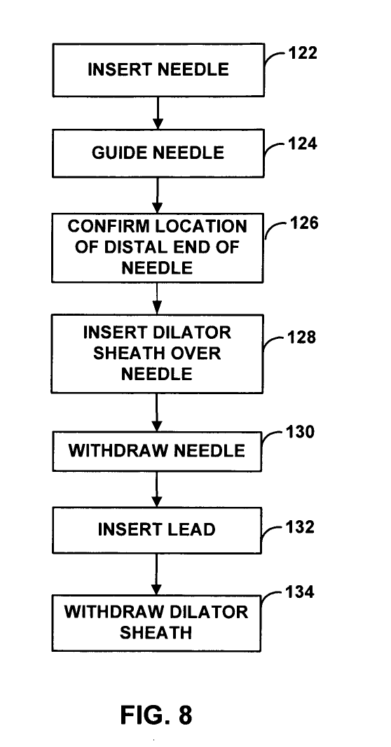

FIG. 8 is a flowchart illustrating one embodiment of implanting lead 14 using dilator sheath 94 rather than dilator assembly 90. However, any implantation procedure utilizing dilator assembly 90 may be modified to utilize only dilator sheath 94. In the illustrated embodiment, needle 70 is inserted into patient 16 (122) and guided to target stimulation site 18 (124). The location of distal end 70B of needle 70 is confirmed using one or more imaging techniques, electrical test signals delivered via needle 70, and/or other appropriate means (126). Once the position of needle 70 is confirmed, dilator sheath 94 is inserted over needle 70 (128). After dilator sheath 94 is properly placed, needle 70 is removed from patient 16 (130). If necessary, additional dilator sheaths of sequentially larger diameter may be inserted over dilator sheath 94 to further dilate the insertion path initially defined by needle 70. Once the insertion path is properly dilated, lead 14 is inserted into patient 16 (132) and positioned proximate to target stimulation site 18. Dilator sheath 94 may then be removed from patient 16 (134).

FIGS. 9A-9C illustrate another embodiment, in which guide wire 101 is used as a guide for dilator assembly 90. Guide wire 101 may be a flexible guide wire, a stiff guide wire, a stylet, or any other appropriate structure. For example, in one embodiment, guide wire 101 is a thin biocompatible stainless steel wire with a diameter. In one embodiment, guide wire 101 may have a diameter around 0.076 cm. As shown in FIG. 9A, after needle 70 is inserted into skin 71 of patient 16 and distal end 70B is positioned near target stimulation site 18, distal end 101B of guide wire 101 may be inserted into lumen 76 of needle 70 along the longitudinal direction of needle 70 (indicated by arrows 136) and distal tip 101C of guide wire 101 may be aligned with distal tip 70C of needle 70.

Needle hub 74 of needle 70 may define an inner lumen (not shown) that is sized to receive guide wire 101, which may help eliminate the need to remove needle hub 74 of needle 70 prior to insertion of guide wire 101. Once guide wire 101 is properly positioned with respect to needle 70, needle 70 may be removed. Guide wire 101, which remains within patient 16 along the insertion path 93 initially defined by needle 70, may serve as a guide for dilator assembly 90. As shown in FIG. 9C, distal end 90B of dilator assembly 90 may be inserted over and around proximal end 101A of guide wire 101 in a manner similar to that described with respect to the insertion of dilator assembly 90 over needle 70.

Guide wire 101 may have markers 105A-105E (shown in FIG. 9B), which mark predetermined locations of guide wire 101. Markers 105A-105E may have properties similar to those described with respect to markers 84A-84E of needle 70 and markers 91A-91E of dilator sheath 94. For example, markers 105A-105E may be radio-opaque or otherwise visible using imaging techniques such as fluoroscopy or X-ray. Additionally, markers 105A-105E may correlate with markers 84A-E on needle and/or markers 91A-E on dilator sheath 94, and a clinician may align at least one marker 105A-E with a correlating marker 84A-E or marker 91A-E to determine the location and/or depth of guide wire 101 within patient 16 or the location of distal end 101 B of guide wire 101 with respect to distal end 70B of needle 70 or distal end 90B of dilator assembly 90.

Guide wire 101 may be removed after dilation of insertion path 93 has been completed. More specifically, after needle 70 has been guided into patient 16 to a desired location proximate to target tissue site 18, guide wire 101 is inserted into needle 70 and advanced to the target tissue site 18. Once guide wire 101 is in place, needle 70 is removed while retaining guide wire 101 at target tissue site 18. In this way, guide wire 101 remains within the insertion path 93 from entry point 92 at skin 71 to target stimulation site 18, which was previously defined by needle 70. Dilator assembly 90 is placed over guide wire 101 to create a path with a diameter sufficient for receiving stimulation lead 14. Once the dilator assembly 90 is in place, guide wire 101 is removed from the dilator assembly 90.

Like needle 70 and dilator assembly 90, guide wire 101 may comprise an electrically insulated body with electrically coupled conductive portions adjacent to proximal end 101A and distal end 101B. In this manner, a trial stimulator may be coupled to proximal end 101A of guide wire 101 in order to send electrical signals to distal end 101B. In this manner, guide wire 101 may be used to conduct test stimulation to assess the efficacy of stimulation prior to implantation of neurostimulation lead 14 and to establish the depth and position of distal end 101B of guide wire 101 with respect to target stimulation site 18. In addition, the electrical test signal sent via guide wire 101 may be used to confirm that distal end 101B of guide wire 101 is generally in the same region as distal end 70B of needle 70 was by, for example, comparing response of patient 16 to the electrical test signals sent via needle 70 and guide wire 101.

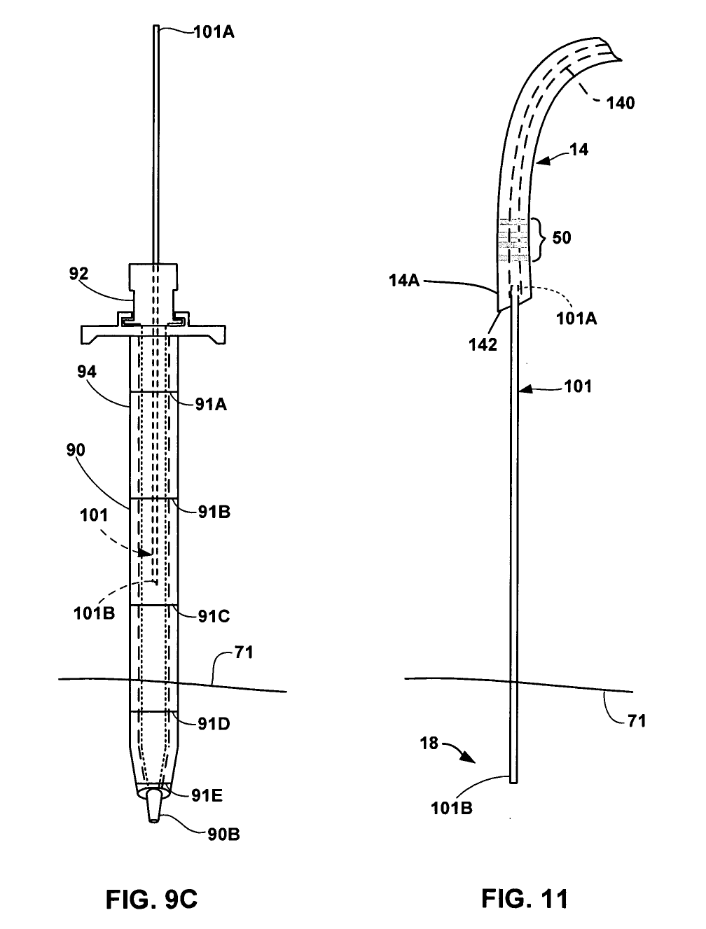

FIG. 10 is a flowchart illustrating an embodiment of implanting lead 14 proximate to target stimulation site 18 utilizing guide wire 101. Needle 70 is inserted into patient 16 (150) and guided to target stimulation site 18 (152). The location of distal end 70B of needle 70 is confirmed using one or more imaging techniques, observing markers 84A-E, electrical test signals delivered via needle 70 or another suitable technique (154). Once the position of needle 70 is confirmed, guide wire 101 is inserted into lumen 76 of needle 70 (156). After guide wire 101 is fully inserted (such that distal end 101B is positioned near distal end 70B of needle 70), needle 70 is removed from patient 16 (158). Next, dilator assembly 90 is inserted over guide wire 101 (160). After dilator assembly 90 is properly positioned proximate to target stimulation site 18 (which may be confirmed via sending an electrical test signal, by visually observing markers 91 A-E, or another suitable technique), guide wire 101 is removed from patient 16 (162). Next, dilator body 92 is removed from dilator sheath 94 and from patient 16 (164), and lead 14 is inserted into dilator sheath 94 and advanced through dilator sheath 94 to target stimulation site 18 (166). Once lead 14 has been properly placed proximate to target stimulation site 18, dilator sheath 94 may be removed from patient 10 (168).

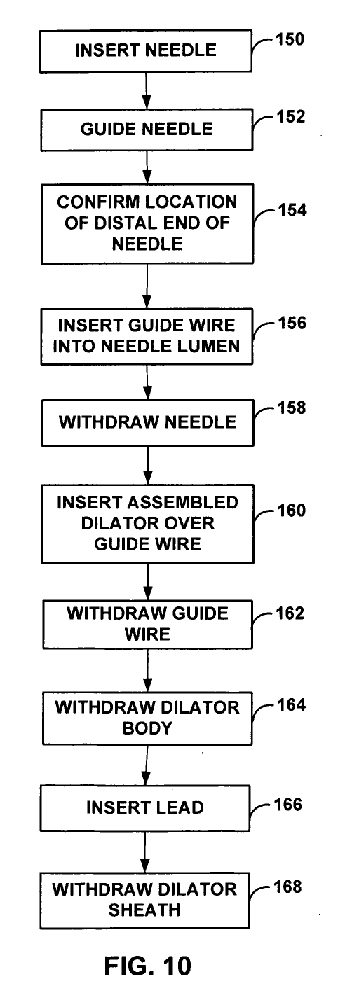

In an alternative embodiment, as shown in FIG. 11, guide wire 101 is inserted into patient 16 through skin 71 to guide stimulation lead 14, and stimulation lead 14 functions as a dilator. In this manner, a separate dilator (e.g., dilator assembly 90 or dilator sheath 94) is not used. More specifically, after needle 70 initially defines the insertion path 93, guide wire 101 is inserted into needle 70. Once guide wire 101 is in the desired location proximate to target tissue site 18, needle 70 is removed from patient 18. In one embodiment, stimulation lead 14 is configured with a centrally located lumen 140 and a pointed tip 142 to help facilitate traversal of stimulation lead 14 through tissue. Distal end 14A of stimulation lead 14 may be inserted over guide wire 101 by allowing proximal end 101A of guide wire 101 to enter lumen 140 of stimulation lead 14. Stimulation lead 14 may be advanced over guide wire 101 to the desired location proximate to target tissue site 18. After electrodes 50 of stimulation lead 14 are located proximate to target tissue site 18, guide wire 101 may be removed from stimulation lead 14.

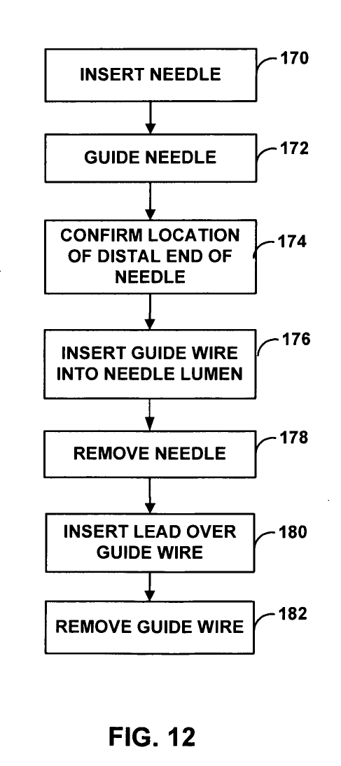

FIG. 12 is a flow chart illustrating an embodiment of the implantation of lead 14 utilizing lead 14 as a dilator. Needle 70 is inserted into patient 16 (170) and guided to target stimulation site 18 (172). The location of distal end 70B of needle 70 is confirmed using one or more imaging techniques, electrical test signals sent via needle 70, and/or other appropriate means (174). Once the position of needle 70 is confirmed, guide wire 101 is inserted into lumen 76 of needle 70 (176). After guide wire 101 is fully inserted, needle 70 is removed from patient 16 (178). Next, lumen 140 of lead 14 is aligned with guide wire 101 and lead 14 is inserted over guide wire 101 (180) such that guide wire 101 is introduced into lumen 140. Lead 14 is advanced to target stimulation site 18. Once lead 14 is properly positioned, guide wire 101 may be removed from patient 16 (182).

In accordance with embodiments utilizing dilator sheath 94 (either individually or as part of dilator assembly 90), FIG. 13 illustrates insertion of lead 14 into dilator sheath 94. Incision 102, which is made to help facilitate insertion of dilator assembly 90, is also shown in FIG. 13. Skin incision 102 may be approximately two centimeters (cm) in length and may be located in the neck of patient 16 lateral to the midline of the spine at the level of the C1 vertebra. In other embodiments, the length of skin incision 102 may be approximately 5 centimeters (cm) in length. The length of skin incision 102 may vary depending on the particular patient and the particular size of dilator sheath 94 because typically, incision 102 is typically sized to receive dilator sheath. At this location, the skin and muscle of patient 16 are separated by a band of connective tissue referred to as fascia.

Although FIG. 13 illustrates incision 102 proximate to the neck of patient 16, in other embodiments and made in a substantially vertical direction, incision 102 may be made at any suitable location and in any suitable direction. For example, as previously discussed, in some embodiments, such as when neurostimulator 12 is implanted near a trapezius muscle of patient 16, a single incision may be made at the implant site for neurostimulator 12.

In FIG. 13, lead 14 is aligned to be introduced into dilator sheath 94 and implanted proximate to occipital region 11 of patient 16 for stimulation of one or more occipital nerves 62, 64, and/or 66 or a trigeminal nerve (not shown). Neurostimulator 12 (FIG. 1) may deliver stimulation therapy to any one or more of lesser occipital nerve 62, greater occipital nerve 64 or third occipital nerve 66 via electrodes disposed adjacent to distal end 14B of lead 14. In alternate embodiments, lead 14 may be positioned proximate to one or more other peripheral nerves proximate to occipital nerves 62, 64, and 66 of patient 16, such as nerves branching from occipital nerves 62, 64 and 66, as well as stimulation of any other suitable nerves throughout the head and neck of patient 16, such as, but not limited to, the trigeminal nerves of patient 16.

Lead 14 may be advanced through dilator sheath 94 and positioned to allow stimulation of target tissue site 18. Lead 14 may also comprise a centrally located lumen (not shown) designed to accept a stylet to assist in the insertion of lead 14. Lead 14 may also have one or more depth markers (not shown) indicate how far lead 14 has been advanced. In one embodiment, one or more depth markers on lead 14 are exposed proximate to proximal end 94A of dilator sheath 94 as lead 14 is inserted. In this manner, one or more depth markers may help ensure that electrodes 50A-50D have exited distal end 94B of dilator sheath 94 but have not advanced too far past distal end 94B (and possibly past target stimulation site 18). When inserting implantable stimulation lead 14, lead 14 is advanced through dilator sheath 94 to target tissue site 18. Lead 14 may also include fixation elements 30. Fixation elements 30 may be restrained by dilator sheath 94 as lead 14 is inserted into patient 16 in order to help prevent premature engagement of fixation elements 30 with surrounding tissue.

Upon placement of lead 14, dilator sheath 94 may be removed from patient 16. When removing dilator sheath 94 from patient 16, care should be taken to avoid displacing stimulation lead 14. Prior to fully removing dilator sheath 94, the position of stimulation lead 14 may be re-verified by utilizing fluoroscopic imaging, applying an electrical signal to evoke a patient response, or using any other suitable technique to ensure that stimulation lead 14 is in the desired location proximate to target tissue site 18.

In some embodiments, the removal of dilator sheath 94 activates fixation elements 30. For example, in the case of tine-like fixation elements 30, the removal of dilator sheath 94 may allow the tine-like elements to expand via elastic force or if fixation elements 30 comprise an adhesive, withdrawal of dilator sheath 94 may expose the adhesive to surrounding fluid or temperature to activate the adhesive. Alternatively, one or more additional steps may be necessary to activate fixation elements 30.

The implantation method described with respect to FIGS. 3-13 may be useful for implantation of stimulation leads 14 and 15 for acute test stimulation or implantation of stimulation lead 14 for chronic stimulation. Typically, the same procedure is used for both applications. Since a chronic stimulation lead, e.g., lead 14, may be inserted without the requirement for a separate test stimulation lead (not shown), the chronic stimulation lead may be placed without positioning repeatability variation. Also, there may be a greater correlation between acute test stimulation and chronic therapy stimulation because the same lead 14 is used to perform both test stimulation and therapy stimulation. Target tissue site 18 may be any area of occipital region 11 intended to achieve a therapeutic effect, such as occipital nerves 62, 62 and/or 66. One way to verify the position of implantable medical lead 14 is to apply an electrical signal to stimulation lead 14 to evoke a motor or sensory response from patient 16. Other ways to verify the location of lead 14 include imaging techniques such as fluoroscopy and x-ray.

Lead 15 may be implanted using a procedure similar to the procedure used to implant lead 14. For example, lead 15 may be implanted using a dilator assembly inserted through incision 102. Alternatively, lead 15 may be inserted using other variations of the implantation process described with respect to lead 14. A separate assembly including a separate needle 70, dilator assembly 90 and/or guide wire 101 from that used to implant needle 14 may be used to implant lead 15.

In some embodiments, therapy system 10 only includes a single lead 14. However, two or more leads may be useful for stimulating more than one target stimulation site, for achieving a greater number of electrode configurations or for achieving bilateral stimulation. In general, bilateral stimulation includes stimulation of two regions of a patient either sequentially or simultaneously. The two regions are typically on opposite sides of midline 9 (FIG. 1) of patient 16, and typically include two branches of a nerve. Bilateral stimulation may include, for example, stimulation of two branches of occipital nerves 62, 64 or 66 (FIG. 13) or the trigeminal nerve that are on opposite sides of the head of patient 16. Bilateral stimulation may also be achieved with a single lead 14, where electrodes of the lead are positioned to span both regions of stimulation. For example, bilateral stimulation of an occipital nerve may be achieved by utilizing a single lead 14 that is placed such that electrodes 50 span both sides of the midline 9 of patient 16 and proximate to the branches of the occipital nerve to be stimulated.

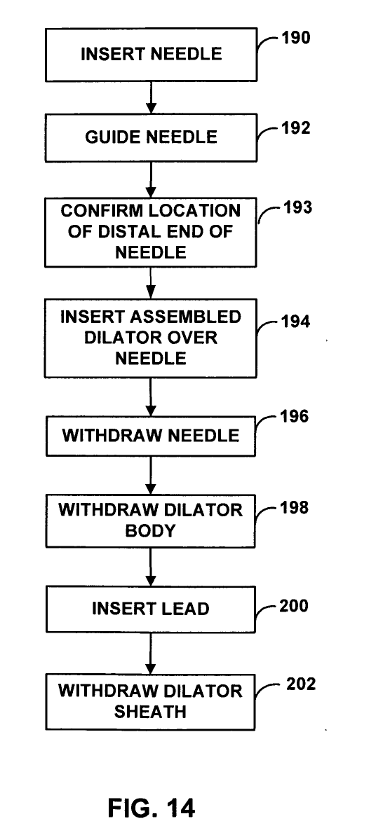

FIG. 14 is a flowchart illustrating one embodiment of implanting neurostimulation lead 14. As described with respect to FIGS. 3-8, variations of the method outlined in FIG. 14 may also be used to implant lead 14. First, needle 70 is inserted into patient 16 (190), and needle 70 is guided to target tissue site 18 (192). The location of distal end 70B of needle 70 may be confirmed (193). For example, trial electrical signals may be sent to distal end 70B of needle 70 to aid in positioning needle 70. If trial electrical signals are sent to distal end 70B of needle 70, a clinician may receive feedback from patient 16 to help determine whether distal end 70B of needle 70 is properly positioned. The patient feedback may indicate, for example, whether the electrical stimulation is felt, whether the electrical stimulation induces paresthesia, whether patient 16 is afflicted by any side effects, and so forth. Alternatively, the clinician may confirm proper placement of the needle by relying on anatomical landmarks (e.g., the C1 vertebra).

Next, dilator assembly 90 is inserted over needle 70 until distal end 90B of dilator assembly 90 is aligned with distal end 70B of needle 70 (194). If necessary, an incision is made at the needle 70 entry point prior to introducing dilator assembly 90 into patient 16. After dilator assembly 90 has been properly placed with respect to distal end 70B of needle 70 as well as target stimulation site 18, needle 70 is withdrawn from patient 16 (196). When dilator assembly is assembled, trial electrical signals may be sent from proximal end 92A to distal end 92B of dilator body 92 to determine the proper location of distal end 92B. During trial stimulation, dilator sheath 94 electrically insulates dilator body 92 such that electrical signals are selectively delivered to distal end 92B of dilator body 92.

Next, dilator body 92 is withdrawn from lumen 100 of dilator sheath 94 (198). After dilator body 92 is removed, lead 14 may be inserted into dilator sheath 94 (200) and advanced through dilator sheath 94 toward distal end 94B of dilator sheath 94. Electrodes 50A-50D of lead 14 are positioned proximate to target tissue site 18. The position of electrodes 50A-50D may be verified by delivery trial electrical signals to electrodes 50A-50D. Once lead 14 is properly positioned, dilator sheath 94 is removed (202). The removal of dilator sheath 94 (202) may activate tine-like fixation elements 30 by allowing fixation elements 30 to expand via elastic force. In other embodiments, one or more additional steps may be required to activate fixation elements 30.

After lead 14 is properly positioned with respect to target stimulation site 18, lead 14 may be coupled to neurostimulator 12. In some embodiments, such as embodiments in which neurostimulator 12 is implanted within patient 16, a clinician may tunnel proximal end 14A of lead 14 to the implant site for neurostimulator 12.

Various embodiments of the invention have been described. These and other embodiments are within the scope of the following claims.

1. A method comprising:
inserting a needle comprising a needle distal end into a patient;
guiding the needle distal end to a target tissue site proximate to at least one of an occipital nerve or a trigeminal nerve of the patient, wherein the needle defines an insertion path through tissue of the patient;
inserting a dilator into the patient through at least a portion of the insertion path;
delivering an electrical test signal to the patient via at least one of the needle or the dilator;
removing the needle from the patient;
inserting an implantable medical lead into the patient through the dilator; and
removing the dilator from the patient.

2. The method of claim 1, wherein the dilator comprises a dilator sheath and a dilator body disposed within the dilator sheath, wherein removing the dilator from the patient comprises:
removing the dilator body from the patient; and
removing the dilator sheath from the patient after the implantable medical lead is advanced through the dilator sheath to the target tissue site.

3. The method of claim 1, further comprising determining a location of the dilator within the patient.

4. The method of claim 3, wherein the dilator comprises a dilator sheath and an electrically conductive dilator body disposed within the dilator sheath, and wherein determining the location of the dilator within the patient comprises applying the electrical test signal to the dilator body.

5. The method of claim 3, wherein the dilator comprises a dilator sheath and a dilator body disposed within the dilator sheath, at least one of the dilator body or the dilator sheath comprising a visible marker, and wherein determining the location of the dilator within the patient comprises locating the visible marker relative to skin of the patient.

6. The method of claim 1, wherein inserting the dilator into the patient through at least the portion of the insertion path comprises inserting the dilator around the needle to introduce the needle into a lumen within the dilator.

7. The method of claim 1, further comprising receiving feedback from the patient in response to the electrical test signal, wherein the feedback indicates at least one of an efficacy of stimulation, a presence of a side effect, or a location of the needle or the dilator within the patient.

8. The method of claim 1, wherein the dilator comprises a dilator proximal portion electrically coupled to a dilator distal portion, and delivering the electrical test signal comprises coupling an electrical stimulator to the dilator proximal portion to deliver the electrical test signal to the dilator distal portion.

9. The method of claim 1, wherein the needle further comprises a needle proximal end that is electrically coupled to the needle distal end, and delivering the electrical test signal comprises coupling an electrical stimulator to the needle proximal end to deliver the electrical test signal to the needle distal end.

10. The method of claim 1, wherein at least one of the needle, the dilator, or the lead comprises a visible marker.

11. The method of claim 10, wherein the visible marker is selected from a group consisting of a radio-opaque marker, a fluoroscopically visible marker, a printed marking or an indentation.

12. The method of claim 1, further comprising determining a location of the needle distal end within the patient.

13. The method of claim 12, wherein determining the location of the needle distal end comprises applying the electrical test signal to the needle.

14. The method of claim 12, wherein the needle comprises a visible marker and determining the location of the needle distal end within the patient comprises locating the visible marker relative to skin of the patient.

15. The method of claim 1, further comprising inserting a guide wire into the patient through the needle to the target tissue site, wherein removing the needle comprises removing the needle from around the guide wire, and advancing the dilator into the patient comprises advancing the dilator around the guide wire.

16. The method of claim 15, and further comprising determining a location of the guide wire within the patient.

17. The method of claim 16, wherein the guide wire comprises an electrically conductive guide wire proximal portion electrically coupled to an electrically conductive guide wire distal portion, and determining the location of the guide wire comprises applying an electrical signal to the guide wire proximal portion to deliver stimulation to the guide wire distal portion.

18. The method of claim 16, wherein the guide wire comprises a visible marker, and determining the location of the guide wire in the patient comprises locating the visible marker relative to skin of the patient.

19. The method of claim 1, wherein inserting the needle into the patient comprises percutaneously introducing the needle proximate to the target tissue site.

20. The method of claim 1, wherein guiding the needle distal end to the target stimulation site comprises positioning the needle substantially transversely across an occipital nerve.

21. The method of claim 1, wherein guiding the needle distal end to the target stimulation site comprises positioning the needle substantially transversely across a trigeminal nerve.

22. The method of claim 1, wherein the needle is a first needle comprising a first needle distal end and defines a first insertion path, the target tissue site is a first target tissue site proximate to a first branch of at least one of the occipital nerve or the trigeminal nerve, the dilator is a first dilator, the electrical test signal is a first electrical test signal, and the implantable medical lead is a first implantable medical lead, and the method further comprises:
inserting a second needle comprising a second needle distal end into the patient;
guiding the second needle distal end to a second target tissue site proximate to a second branch of at least one of the occipital nerve or the trigeminal nerve, wherein the second needle defines a second insertion path through tissue of the patient;
inserting a second dilator into the patient through at least a portion of the second insertion path;
delivering a second electrical test signal to the patient via at least one of the second needle or the second dilator;
removing the second needle from the patient;
inserting a second implantable medical lead into the patient through the second dilator; and
removing the second dilator from the patient.

23. The method of claim 22, further comprising positioning the first and second implantable medical leads within the patient to achieve bilateral stimulation of at least one of the occipital nerve or the trigeminal nerve.

24. The method of claim 1, further comprising positioning the implantable medical lead across a midline of the patient to achieve bilateral stimulation of at least one of the occipital nerve or the trigeminal nerve.

25. The method of claim 1, further comprising coupling the lead to a medical device, the medical device being configured to deliver a therapy to the target tissue site via the lead.

26. The method of claim 1, wherein the lead comprises one or more electrodes proximate to a lead distal end and a fixation element proximate to the electrodes to substantially fix the lead proximate to the targets therapy delivery site.

27. A method comprising:
inserting a needle comprising a needle distal end into a patient;
guiding the needle distal end to a target tissue site proximate to at least one of an occipital nerve or a trigeminal nerve of the patient, the needle defining an insertion path from skin of the patient to the target tissue site;
inserting a guide wire through the needle to the target therapy delivery within the patient;
removing the needle from the patient;
inserting an implantable medical lead into the patient through the insertion path;
removing the guide wire from the patient; and
delivering an electrical test signal to the patient via at least one of the needle or the guide wire.

28. The method of claim of 27, wherein at least one of the needle, the guide wire, or the lead comprises a visible marker.

29. The method of claim 27, wherein the needle comprises a needle proximal end electrically coupled to the needle distal end, and delivering the electrical test signal comprises coupling an electrical stimulator to the needle proximal end to deliver the electrical test signal to the needle distal end.

30. The method of claim 27, wherein the guide wire comprises a guide wire proximal portion electrically coupled to a guide wire distal portion, and wherein delivering the electrical test signal comprises coupling an electrical stimulator to the guide wire proximal portion to deliver the electrical test signal to the guide wire distal portion.

31. The method of claim 27, further comprising receiving feedback from the patient in response to the electrical test signal, wherein the feedback indicates at least one of an efficacy of stimulation, a presence of a side effect, or a location of the needle or the guide wire within the patient.

32. The method of claim 27, further comprising inserting a dilator into the patient around the guide wire and through at least a portion of the insertion path.

33. The method of claim 27, wherein the implantable medical lead defines an inner lumen, and inserting the implantable medical lead into the patient through the insertion path comprises introducing the guide wire into the inner lumen of the implantable medical lead.

34. A method comprising:
inserting a needle into a patient;
guiding a needle distal end of the needle to a target tissue site proximate to at least one of an occipital nerve or a trigeminal nerve of the patient, the needle defining an insertion path through tissue of the patient;
dilating the insertion path;
delivering an electrical test signal to the patient via the needle;
removing the needle from the patient; and
inserting an implantable medical lead into the dilated insertion path.

35. The method of claim 34, wherein dilating the insertion path comprises:
inserting a dilator comprising a dilator distal end through the insertion path;
guiding the dilator distal end to the target tissue site through at least a portion of the insertion path, wherein the dilator is configured to receive the implantable medical lead; and
removing the dilator from the patient.

36. The method of claim 35, wherein the dilator is inserted through the insertion path around the needle, the needle being received in a dilator lumen of the dilator.

37. The method of claim 34, wherein dilating the insertion path comprises:
inserting a guide wire into a first lumen defined by the needle prior to removing the needle from the patient;
aligning a second lumen defined by the implantable medical lead with the guide wire, the second lumen being configured to receive the guide wire; and
inserting the lead around the guide wire, wherein the implantable medical lead dilates the insertion path.

38. The method of claim 34, wherein the needle is a first needle and defines a first insertion path, the needle distal end is a first needle distal end, the target tissue site is a first target tissue site, the electrical test signal is a first electrical test signal, and the implantable medical lead is a first implantable medical lead, and the method further comprises:
inserting a second needle into the patient;
guiding a second needle distal end of the second needle to a second target tissue site on an opposite side of a midline of the patient from the first therapy delivery site, the needle defining a second insertion path through tissue of the patient;
dilating the second insertion path;
delivering a second electrical test signal to the patient via the second needle;
removing the second needle from the patient; and
inserting a second implantable medical lead into the dilated second insertion path.
